(19) United States
(12) Patent Application Publication
(10) Pub. No.: US 2017/0231660 A1
(43) Pub. Date: Aug. 17, 2017

(54) DEVICE FOR INDUCING OR CORRECTING MANDIBLE GROWTH

(71) Applicant: Yoomed, Montpellier (FR)

(72) Inventor: Pierre Bonnaure, Rennes (FR)

(21) Appl. No.: 15/518,692

(22) PCT Filed: Nov. 3, 2015
(86) PCT No.: PCT/FR2015/000201

§ 371 Date: Apr. 12, 2017

(30) Foreign Application Priority Data
Nov. 3, 2014 (FR) 1460552

(51) Int. Cl. A61B 17/66; A61F 5/56

(52) U.S. Cl. CPC A61B 17/663; A61F 5/56

(57) ABSTRACT

The present invention relates to a device for inducing or correcting mandible growth of a newborn.

The device (1) according to the invention is characterized in that it comprises:
a support (100) for supporting at least one part of the rear surface of the body of the newborn, including the head, preferably in the inclined supine position,
at least one extra-oral means (104, 105) for exerting a pushing force on each of the two rising branches of the mandible by means of at least one transdermal bone anchoring device (107, 108) in said branch,
at least one extra-oral means (102, 103) for exerting a traction or pushing force on each of the two horizontal branches of the mandible by means of at least one transdermal bone anchoring device (106) in said branch, said extra-oral means (102, 103, 104, 105) for exerting forces being linked to the support (100) by linking means (109, 110) and to said transdermal bone anchoring devices by connection means (113, 114, 115).

The present invention relates to a device for inducing or correcting mandible growth.

Among the pathologies related to a mandible growth defect, Pierre Robin syndrome is a pathology causing, from birth, severe feeding, breathing and heart rate regulation anomalies. The frequency of this pathology is one out of 8,000-10,000 newborns.

Pierre Robin syndrome, more precisely Pierre Robin sequence, is characterized by the presence at birth of an orofacial morphological triad including three anomalies of the mouth and the face: retrognathism, glossoptosis and the presence of a posterior median velopalatal cleft. Retrognathism designates a lower jaw smaller than normal with a retreated chin. Glossoptosis defines the possibility for the tongue to fall backwards in the throat, and the posterior median velopalatal cleft represents the absence of closing at the back of the palate. From a phenotypic point of view, the appearance of the face at birth is very typical, showing a profile referred to as “bird profile.”

A morphological anomaly without respiratory distress is seen at stage I of Robin syndrome. Respiratory distress seen at stage II reveals a respiratory obstruction which can also cause a suction-deglutition disorder, vagal hypertonia and gastroesophageal reflux. A serious form of the disease occurs at stage III, in which respiratory failure is very severe and the consequences described in relation to stage II are greatly amplified.

ORL disorders, with important hearing loss, dental disorders, speech disorders, intellectual disability if oxygen deficiency during the first weeks of life was important, skeletal disorders, or other disorders affecting more distant organs such as the eyes, heart, genitalia or brain, are seen in children suffering from this syndrome at more advanced ages, depending on the above-mentioned stages and the management carried out.

Different approaches are considered today for managing respiratory disorders resulting from a mandible growth defect. These approaches are generally established depending on the stage of the disease.

The simplest medical therapy is to place the newborn in ventral decubitus. In this position, the tongue projects forward, thus clearing the upper airways. Nasopharyngeal intubation by means of a cannula can also be considered. This method allows the passage of air through the cannula and the forward propulsion of the tongue. Endotracheal intubation or non-invasive ventilation through a mask is also carried out.

These methods are effective in improving ventilation in children, but they do not induce or restore mandible growth.

The surgical therapies currently carried out are based on mandibular traction. A main difficulty when managing mandibular traction is related to the fact that newborns have no teeth. As a result, it is impossible to use mandibular advancement orthosis using a dental support for forcing mandibular advancement. These mandibular advancement processes are therefore carried out on children with teeth starting at 5-6 years old. In many cases, the respiratory disorders are so severe and sources of secondary and worrying pathologies that Robin syndrome management cannot wait until this more advanced age. Furthermore, mandible growth is remarkable in newborns compared to that seen in 6-year-old children, which allows for quick and effective action. Consequently, management at this stage of development would seem to be a suitable approach.

The first surgical therapy in infants was developed in 1957 by Stellmach and then abandoned due to great consequences on the temporomandibular joint. In 2011, another surgeon, Barciliero, described his work on mandibular traction by means of threads inserted around the mandible, in the parasymphyseal region. The two threads parallel to each other extend on the outside in a symmetrical traction direction on both sides to a point generally fixed to the bed and holding a weight. This method allows inducing mandible sagittal growth acceleration. A similar method in which the threads are replaced by a plate which is fixed to the mandible by means of cerclage is also used. Although effective for managing respiratory disorders, the persistence of mandibular retrognathia is observed.

Surgical therapy currently performed that seeks to induce or restore mandible growth is based on mandibular traction and osteosynthesis. This is mandibular osteogenic distraction. It is performed by means of a device known as a distractor. This method allows for a gradual forward elongation of the mandible. As a consequence, pharyngeal diameter is increased and the tongue projects forward. Respiratory obstructions are suppressed and the lack of mandible growth is corrected. The method consists of performing osteotomy of the branches of the mandible and then placing the fasteners or screws of the distractor on both sides of the bone section zone. The separation between the two bone sections is kept by means of a distraction screw. This screw is regularly actuated for elongating the osteotomy zone 0.5 to 1 mm per period. Bone synthesis and consequently elongation of the mandible are observed.

Although this method provides satisfactory results, it requires a surgical action for placing the distractor, which can be qualified as heavy when applied to a newborn.

Published international patent document number WO 2012/156247 describes a device for the treatment and alleviation of obstructive respiratory sleep disorders consisting of a support for the body of a patient including a headrest, a seat and a support for the rib cage. When the patient sits on the support, his/her chest is held inclined forward at an angle of less than 90° , generally between 45 and 60° . Orthoses made up of thermoformed grooves are provided to be arranged on the maxilla and the mandible and linked, by means of branches projecting outside the oral cavity, to the frame of the support in order to exert a traction force on the mandible and/or on the maxilla. Although effective, this device is not suitable for the morphology of newborns since the use of thermoformed grooves cannot be considered in newborns without dentition, as previously mentioned. Furthermore, the device is suitable for the morphology of children old enough to sit down.

One objective of the present invention is to propose a device for inducing or correcting mandible growth which can be used in newborns, from birth, without the presence of dental arches, being of a simple design and use and for a very short period of time, which only requires a brief surgical operation, which is the least invasive and leaves the smallest negative impression possible and allows exerting pushing or traction forces on the mandible, from an extra-oral point, that is, without resting on the maxillary bone, or another bone or body element of the newborn so as to prevent resulting deformations.

To that end, the present invention relates to a device for inducing or correcting mandible growth of a subject such as a newborn, characterized in that it comprises:
a support for supporting at least one part of the rear surface of the body of the subject, including the head, in the supine position,
at least one extra-oral means for exerting a traction or pushing force on each of the two rising branches of the mandible by means of at least one transdermal bone anchoring device in said branch,
at least one extra-oral means for exerting a traction or pushing force on each of the two horizontal branches of the mandible by means of at least one transdermal bone anchoring device in said branch,
said extra-oral means for exerting forces being linked to the support by linking means and to said transdermal bone anchoring devices by connection means.

According to a preferred embodiment of the invention, the extra-oral means are provided for exerting pushing forces.

According to another preferred embodiment of the invention, the support is provided for positioning the subject in the inclined supine position.

In the sense of the invention, extra-oral means refers to means included in the device according to the invention and not intended for being inserted in the oral cavity of the subject in whom the device is applied, as opposed to intra-oral distractors known in the prior art.

The working principle of the device according to the invention is based on continued and gradual growth only by the osteogenesis reaction of the mandible of the infant. Unlike distraction devices used today, cutting the branches of the mandible before placing the device is not necessary. It uses extra-oral means suitable for exerting pushing or traction forces on the branches of the mandible resting on the device itself, particularly on the support, and not on any bone of the subject. Indeed, such a practice would be harmful in that it would cause bone deformations. Note also the absence of any anchoring to the maxilla, although such an anchoring could be considered depending on the malformation or pathology to be treated.

The device according to the invention allows acting on very young children, from birth, in order to benefit from a maximal growth rate, and even though facial deformations or malformations are even minor. Treatment related to the device can be very short since it can extend over a few weeks, for example two months, and is shown to be very effective.

The device according to the invention allows treating children during the action of growth hormones, that is, mainly while they are sleeping.

The device according to the invention allows acting exclusively on the mandible.

The device according to the invention allows acting independently on the rising branch or the horizontal branch of the mandible since each branch is related to at least one extra-oral means for exerting a force. This also allows modifying the inclination between the rising branch and the horizontal branch.

Finally, the device according to the invention allows putting in place a scarcely invasive treatment compared to the current treatment by mandibular distraction.

Preferably, a transdermal bone anchoring device is a transdermal implant suitable for being osteointegrated. Any other means that allow anchoring the device to the mandible bone is obviously considered.

Preferably, the device comprises two extra-oral means provided, one for exerting a pushing force on the right rising branch of the mandible by means of at least one transdermal bone anchoring device in said right branch, the other one for exerting a pushing force on the left rising branch of the mandible by means of at least one transdermal bone anchoring device in said left branch and two extra-oral means for exerting a pushing force on the two horizontal branches of the mandible by means of at least one transdermal bone anchoring device in the joining zone of said horizontal branches at the level of the mandibular symphysis.

In that preferred embodiment of the invention, the device acts by means of three bone anchoring sites so as to ensure independent growth in the space of the mandibular branches. Each extra-oral means for exerting a force on a rising branch of the mandible is linked to that branch by means of at least one transdermal bone anchoring device, such as an osteointegrated transdermal implant. One bone anchoring site will be found on the right rising branch and another one on the left rising branch of the mandible. However, the two extra-oral means for exerting their force on the horizontal branches of the mandible are linked to said branches by means of at least one transdermal bone anchoring device, preferably only one, which is osteointegrated in the same zone, that of the chin. The device acts nevertheless on the four branches of the mandible.

Advantageously, each extra-oral means for exerting pushing forces comprises an elastic means, such as a spring actuator, or even a gas cylinder or actuator. The pushing means is preferably a spring actuator the spring of which works under compression, since a spring with a specific stiffness suitable for the application can be chosen.

Preferably each extra-oral means for exerting a pushing force is a spring actuator consisting of a cylinder fixed on the support by said linking means and housing a rod and its piston, a compression spring being housed in the cylinder so as to apply a force suitable for causing an end of said rod provided to be connected to a transdermal bone anchoring device to come out, a blocking means, such as a screw passing through the wall of the cylinder, being provided for blocking the rod in a specific position.

The spring actuators are linked to the device and their rods are linked to the free ends of the transdermal bone anchoring devices in the branches of the mandible on which these actuators therefore exert their forces.

Advantageously, the device comprises adjusting means for adjusting intensities and directions of the forces exerted by the extra-oral means on the branches of the mandible. Such means consist of, for example, rotation axes, translation axes, or any other means allowing the movement of the extra-oral means in space.

Indeed, depending on the physiognomy of the subject to be treated and the correction to be provided to the mandible, the device needs to be able to be adapted. Before connecting the device to the subject, a trajectory in space can be defined which each transdermal bone anchoring device must follow, over the duration of the treatment, in order to remodel in a non-symmetrical manner the left/right balance of the mandible of the infant. The device according to the invention allows performing three-dimensional corrections on independent points in space acting on the whole jaw.

Advantageously, the linking means between the support and the means for exerting pushing forces consist of two wings extending forward from the support and intended for flanking the head of the subject, said wings being fixed to the support on pivot pins making them suitable for pivoting between a so-called open position in which they clear the access to the support for placing the head of the subject therein and a so-called closed position in which they allow fixing the extra-oral means for exerting traction or pushing forces on the transdermal bone anchoring devices.

Therefore, the device according to the invention comprises two symmetrical, side mechanical parts which can be retracted by tilting, and each of which includes two independent elements ensuring a specific pushing on the horizontal and rising branches, respectively.

Preferably, the connection means for connecting the actuators to the transdermal bone anchoring devices in the rising branches of the mandible comprise rods which extend perpendicularly to the axis of the actuator cylinders and which are provided at their free ends with ball joints.

Preferably, the connection means between the transdermal bone anchoring device or devices for the anchoring thereof to the mandibular symphysis and the extra-oral means intended for exerting a pushing force on the horizontal branches of the mandible comprise a flexible arcuate strip extending from one of these extra-oral means to the other, said strip having anchoring means at the free end of said bone anchoring devices.

Advantageously, the device comprises guides for positioning and orienting the head of the subject against the support, such that each transdermal bone anchoring device can be connected with the means for exerting a pushing force for which it is intended.

Particularly, the device comprises guides for positioning and orienting the head of the subject against the support and between the wings, such that each transdermal bone anchoring device can be connected with the connection means for which it is intended, the guides consisting of two rods, each one assembled on a wing and made suitable for sliding with respect to that wing when it is in the so-called closed position until the end of the rod slightly enters the auditory canal of the subject, and of a third rod suitable for sliding with respect to the support from the top end of that support until the end of the rod abuts against the apex of the head of the subject.

The positioning guides allow placing the free ends of the transdermal bone anchoring devices facing the connection means for the connection thereof to the means for exerting forces.

Advantageously, the support is in the form of a bassinet, cradle, bed or baby rocker, consisting of a resting surface for the whole rear surface of the subject. Since the support is mainly intended for infants, it will have the typical shape of those supports generally provided for them. Particularly, it will have a surface provided with a thickness and made in a material ensuring suitable comfort.

The features of the invention mentioned hereinabove, as well as others, will become more evident after reading the following description of an exemplary embodiment, said description being made in relation to the attached drawings, in which:

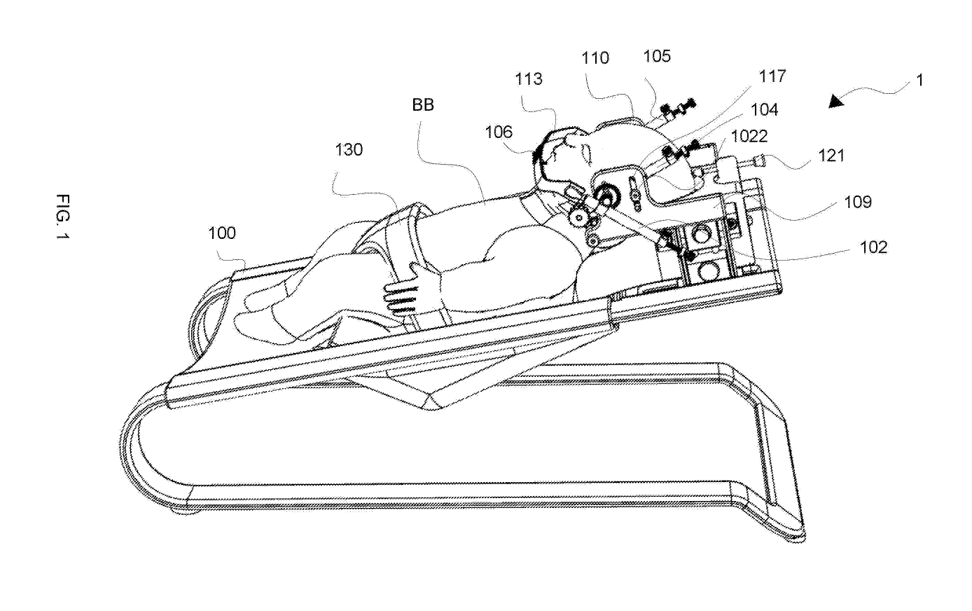

FIG. 1 shows an overview of a device according to the invention according to one embodiment of the invention, in which the subject to be treated is placed,

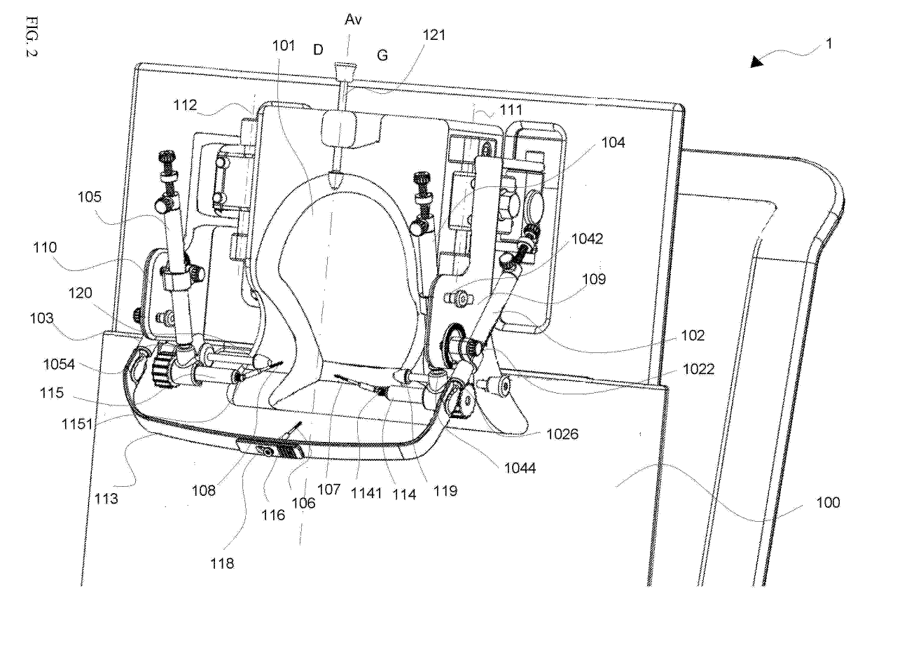

FIG. 2 shows a perspective view of a portion of a device according to one embodiment of the invention, in which the subject to be treated is not placed,

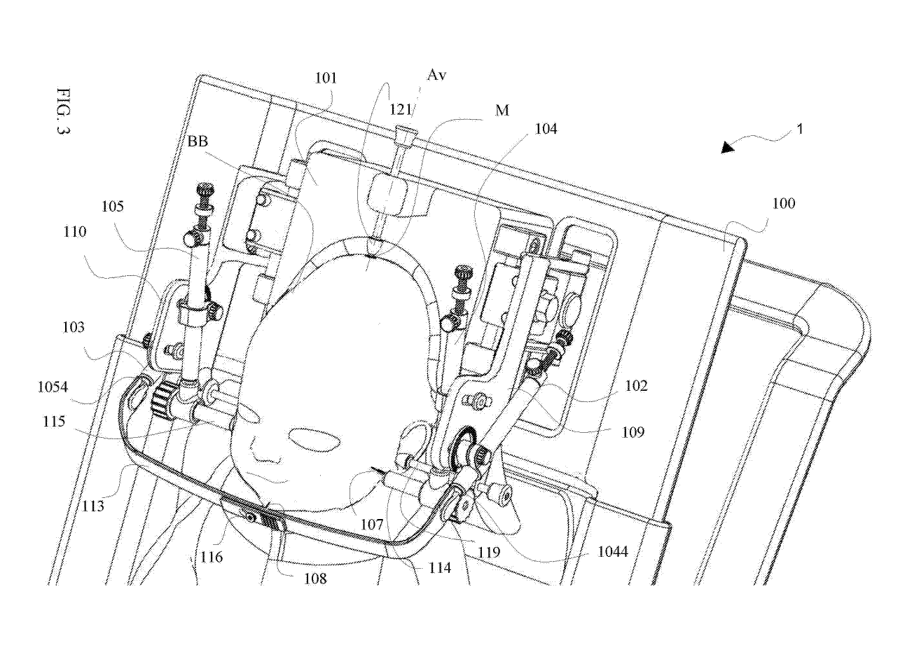

FIG. 3 shows a perspective view of a portion of a device according to one embodiment of the invention, in which the subject to be treated is placed,

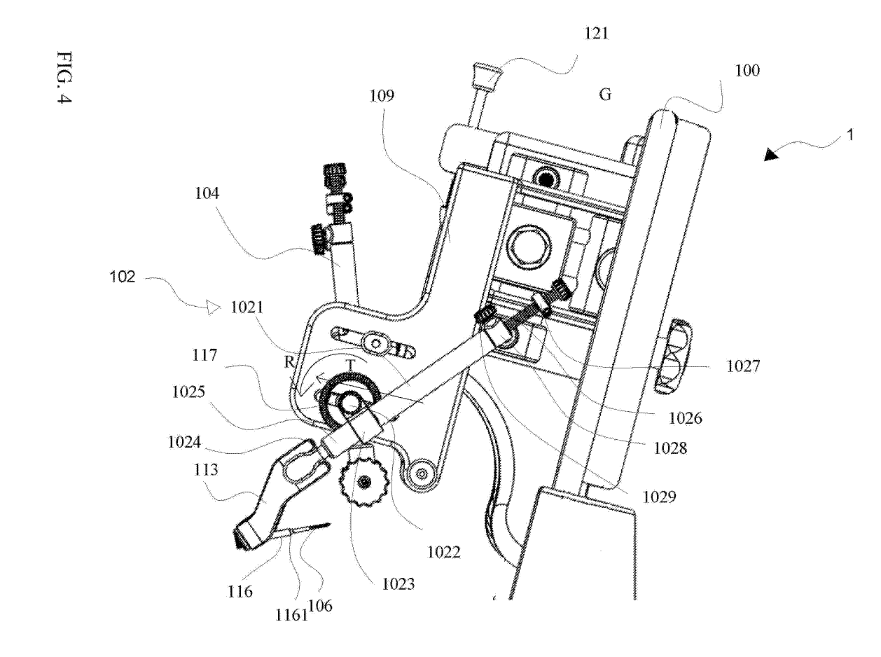

FIG. 4 shows a side profile view of a portion of a device according to one embodiment of the invention, in which the subject to be treated is not placed.

FIG. 1 shows an overview of a device 1 according to the invention in which a subject BB, such as a few weeks old infant, has been placed to be treated therein. The device 1 comprises a support 100 in the shape of a baby rocker on which the subject BB is placed laying on his/her back, preferably in an inclined position. Preferably, the subject BB is inclined according to an angle of about 30° with the ground. Since the device is essentially provided for an infant, the support 100 will be designed for allowing an extended position for the baby in all cases on his/her back. One part of the support 100 has an accommodation 101 for receiving the rear surface of the head of BB and participate in his/her comfort. It can be, for example, a pillow attached to the support 100. A holding strap 130 is optionally provided for ensuring that the subject BB is held extended on the support 100.

FIG. 2 shows the device 1 in the absence of the subject BB. The device 1 is symmetrical on both sides of its vertical central axis Av.

The device 1 comprises four extra-oral means 102, 103, 104 and 105 for exerting pushing forces on the four mandibular branches, respectively, by means of transdermal bone anchoring devices, such as osteointegrated transcutaneous implants 106, 107, 108.

The extra-oral means 102, 103, 104 and 105 for exerting forces on the mandible are outside the oral cavity of the subject when he/she is being treated (FIG. 3). In currently used mandibular distraction devices, the bone is placed under stress by means of a distractor inside the oral cavity and even the bone cavity. The extra-oral means 102, 103, 104, 105 are linked to the support 100 by linking means 109, 110 described below.

The transdermal implants 106, 107, 108 are represented in FIG. 2 connected to the device 1, but they are in fact implanted by means of surgery in the branches of the mandible before placing the subject BB on the device 1, and before treating him/her. The implant 106 is at the level of the axis of symmetry Av of the device 1 and is intended for being osteointegrated at the level of the mandibular symphysis, the zone of convergence of the horizontal branches of the mandible. The implant 107 is to the left of the axis of symmetry Av and is intended for being osteofixed in the left rising branch of the mandible. The implant 108 is to the right of the axis of symmetry Av and is intended for being osteofixed in the right rising branch of the mandible.

The extra-oral means 104 and 105 are intended for exerting pushing forces on the left and right rising branches, respectively, of the mandible of the subject. The extra-oral means 102 and 103 are intended for exerting pushing forces on the left and right horizontal branches, respectively, of the mandible of the subject. As a consequence, the extra-oral means 102 and 104 are arranged to the left of the axis of symmetry Av of the device and the extra-oral means 103 and 105 are arranged to the right of the axis of symmetry Av of the device 1. As for the subject BB, he/she is to be placed on the central axis Av of the device 1 as can be seen in FIG. 1.

The pushing forces on the branches of the mandible are exerted from the device 1 since the extra-oral means rest thereon and not on the subject to be treated. For example, the forces can be supported from the support 100 at the back of the head. The principle of treatment consists of defining a trajectory in space which each point of the transdermal implant 106, 107, 108 must follow in order to obtain a growth which remodels the mandible. The operating dimensions are calculated from a reference point located on the device. In addition to its function as an ergonomic guide for the child placing him/her in the best possible position, the support 100 has the function of ensuring the starting point (point “zero”) of the chain of the operating dimensions of the device. It shows the kinematic and mechanical reference of the device. The ensemble of the elements, specifically of the extra-oral means, can be adjusted in function and in dimension from that point. To that end, the extra-oral means 102, 103, 104 and 105 are linked to the support 100 by linking means. In the preferred embodiment of the invention and as depicted in FIGS. 1 to 3, the linking means consist of a pair of wings, or leaves, 109, 110 assembled on the support 100 on pivot pins 111, 112, respectively, on both sides of the axis of symmetry Av. The pivot pins can be established by means of hooks, hinges, pin hinges or any other means known to the person skilled in the art.

The wing 109 supports the extra-oral means 102 for exerting a pushing force on the left horizontal branch of the mandible of the subject BB as well as the extra-oral means 104 for exerting a pushing force on the left rising branch (FIGS. 1 and 3). The wing 110 supports the extra-oral means 103 for exerting a pushing force on the right horizontal branch of the mandible of the subject BB as well as the extra-oral means 105 for exerting a pushing force on the right rising branch (FIGS. 1 and 3).

The wings 109, 110 are provided for flanking the head of the subject BB. In addition, they are provided for pivoting between a so-called open position in which they clear the access to the support 100, particularly for placing the head of the subject BB therein, and a so-called closed position in which they allow connecting the extra-oral means 102, 103, 104, 105 to the transdermal implants 106, 107, 108.

Therefore, the device 1 comprises two symmetrical, side mechanical parts which can be retracted by tilting and each of which includes two independent means ensuring a specific pushing on the horizontal and rising branches, respectively.

The extra-oral means 102 for exerting a pushing force is shown in detail in FIG. 4. It consists of a cylinder 1021 which is fixed to the wing 109 on a rotation shaft 1022 by means of a flange 1023. Inside the cylinder 1021 a rod 1026 and its piston (not shown) can slide freely (FIG. 4). An end 1024 of the rod comes out at the end 1025 of the cylinder 1021. This end 1025 is provided with a connection means 113 for the connection thereof with the implant 106. A compression spring is housed inside the cylinder 1021 so as to apply a force suitable for causing the end of the rod 1024 to come out at the end 1025 of the cylinder 1021. A blocking means 1029, such as a screw passing through the wall of the cylinder 1021, is used for blocking the rod 1026 in a chosen position. A nut 1027 is screwed on the part of the rod 1026 which comes out at the other end 1028 of the cylinder. This nut 1027 acts as a stop to the advancement of the end 1024 of the rod which must be connected to the implant 106.

The pushing means 102 are preferably a spring actuator, since a spring with a specific stiffness suitable for the application can be chosen. In practice, mandible growth is less than 2 cm. Depending on the chosen spring, an almost constant pushing force can be obtained. However, in a variant of the invention, a gas actuator can be absolutely suitable.

The previous description in relation to the extra-oral means 102 applies to the extra-oral means 103, 104 and 105 since they are preferably identical to the means 102.

The connection means for connecting the actuators 104 and 105 to the transdermal implants 107 and 108, respectively, include rods 114 and 115 which extend from the ends 1044 and 1054 of the actuators and perpendicularly to the axis of those actuators and are provided at their free ends with ball joints 1141, 1151 (FIG. 2, FIG. 3). The ball joints allow for the connection between the implants and the actuators. Indeed, while placing the implants in the mandible, they may not be implanted exactly at the expected site. Consequently, the ball joint allows correcting the separation distance between the expected implantation site and the actual implantation site.

The connection means for connecting the actuators 102 and 103 to the transdermal implant 106 include a flexible arcuate strip 113. It extends from the actuator 102 to the actuator 103 and is placed facing the chin of the subject BB when he/she is placed on the support 100 (FIG. 3). The flexibility is required to allow small mandible suction movements for the subject. A rod 116 is also provided. One of its ends is linked to the implant 106 by means of a ball joint 1161 and its other end is housed in a housing 118 performed in the arcuate strip 113. This rod 116 remains preferably connected to the implant 106 such that an offset is arranged in the housing 118 so as to insert the end of the rod 116 in that offset and is then caused to slide in a narrower zone of the housing for the blocking thereof.

The pushing forces are exerted on the branches of the mandible so as to obtain mandible growth in three dimensions. Generally, the force exerted on each rising branch will be directed forwards and downwards. The force exerted on each horizontal branch will be exerted forwards.

The directions of the pushing forces exerted by each extra-oral means 102, 103, 104, 105 can be adjusted by adjusting means. The adjusting means include, specifically, adjusting means for adjusting the position of the axes of the springs of the actuators 102 to 105 in space. For example, concerning the actuator 102, the adjusting means include the rotation shaft 1022 on which the cylinder is fixed on the wing 109. Thanks to this rotation shaft 1022, the axis of the spring can be moved by rotation in a plane parallel to that of the wing 109 (arrow R, FIG. 4). This plane is also substantially parallel to the side face of the branch of the mandible on which the pushing force must be exerted. Therefore, the adjusting means allow the orientation of the axis of the spring of each actuator with respect to the branch of the mandible on which that spring must exert a pushing force.

The adjusting means also include a slot 117 made in the wing 109 and in which the rotation shaft 1022 can be moved (arrow T, FIG. 4), in a plane parallel to that of the wing 109. This plane is also substantially parallel to the side face of the branch of the mandible on which the pushing force must be exerted. These adjusting means also allow adapting the device to the morphology of the subject so as to allow the connection with the osteointegrated implants. Identical adjusting means are provided with respect to the actuators 103, 104 and 105.

The intensity and direction of the force exerted by each of the actuators are determined and adjusted at the beginning of each treatment for each subject. Therefore, when the subject is removed from the device 1 for the purpose of changing or feeding, and then placed therein again, the forces which are applied anew on the mandible are identical to those that were applied before he/she was removed from the device.

When placing the subject BB, the wings 109 and 110 are placed in an open position and therefore each of them pivots in a direction opposite the axis Av. The head of the subject BB is placed against the housing 101 of the support 100. The subject is in an extended position on his/her back against the support 100 (FIG. 1). The wings 109 and 110 are tilted by pivoting in an inverse direction into a so-called closed position in which they flank the head. p In order to correctly place the free ends of the transdermal implants 106, 107, 108 facing the connection means 114, 115, 116 for the connection thereof to the actuators 102, 103, 104, 105, the device 1 comprises guides for positioning and orienting the head of the subject BB against the support 100 and between the wings 109 and 110. These guides consist of two rods 119, 120, respectively assembled on the wings 109 and 110 and suitable for sliding with respect to these wings when they are in the so-called closed position until the end of each rod slightly enters the auditory canal of an ear of the subject (FIG. 3). In addition, a third rod 121 is provided. It is suitable for sliding with respect to the support 100 from the top of the support until the end of the rod 121 abuts against the apex of the head of the subject where a target M in the shape of a mark or tattoo is drawn. Finally, the ball joints 1141, 1151, 1161 also allow for the connection of the transdermal implants 106, 107, 108 which are connected to the means 102, 103, 104, 105.

Once the subject is placed, the transdermal implants 106, 107, 108 are connected to the means 102, 103, 104, 105 exerting the forces via the connection means described above. The rods 119, 120, 121 are moved so as not be in contact with the subject.

As mentioned above, the absence of any anchoring to the maxilla can be noted, although such an anchoring could be considered depending on the malformation or pathology to be treated. In such case, means for exerting forces on the maxilla would be identical to those described in relation to the mandible (102, 103, 104, 105).

The placing of the subject and the removing thereof are simple and they are performed as many times needed depending on the typical daily care of the subject, such as breastfeeding, bathing and cuddling.

1. A device (1) for inducing or correcting mandible growth of a newborn subject, characterized in that it comprises:
a support (100) for supporting at least one part of the rear surface of the body of the subject, including the head, in the supine position,
at least one extra-oral means (104, 105) for exerting a pushing force on each of the two rising branches of the mandible by means of at least one transdermal bone anchoring device (107, 108) in said branch,
at least one extra-oral means (102, 103) for exerting a traction or pushing force on each of the two horizontal branches of the mandible by means of at least one transdermal bone anchoring device (106) in said branch,
said extra-oral means (102, 103, 104, 105) for exerting forces being linked to the support (100) by linking means (109, 110) and to said transdermal bone anchoring devices by connection means (113, 114, 115).

2. The device (1) according to claim 1, characterized in that the extra-oral means (102, 103, 104, 105) are provided for exerting pushing forces.

3. The device (1) according to claim 2, characterized in that the device comprises two extra-oral means (104, 105), one (105) for exerting a pushing force on the right rising branch of the mandible by means of at least one transdermal bone anchoring device (107) in said right branch, the other (104) for exerting a pushing force on the left rising branch of the mandible by means of at least one transdermal bone anchoring device (108) in said left branch and two extra-oral means (102, 103) for exerting a pushing force on the two horizontal branches of the mandible by means of at least one, transdermal bone anchoring device (106) in the joining zone of said horizontal branches at the level of the mandibular symphysis.

4. The device (1) according to claim 2, characterized in that each of the extra-oral means (102, 103, 104, 105) for exerting pushing forces comprise one elastic means.

5. The device (1) according to claim 2, characterized in that each extra-oral means (102, 103, 104, 105) for exerting a pushing force consists of a spring actuator consisting of a cylinder (1021) fixed on the support (100) by said linking means (109, 110) and housing a rod (1026) and its piston, a compression spring being housed in the cylinder (1021) so as to apply a force suitable for causing an end (1024) of said rod (1026) provided to be connected to at least one transdermal bone anchoring device (106) to come out, a blocking means, being provided for blocking the rod (1026) in a specific position.

6. The device (1) according to claim 1, characterized in that the device comprises adjusting means (1022, 117) for adjusting the intensity and the direction of the force exerted by each extra-oral means (102, 103, 104, 105).

7. The device (1) according to claim 2, characterized in that the linking means (109, 110) between the support and the means (102, 103, 104, 105) for exerting pushing forces consist of two wings extending forward from the support and intended for flanking the head of the subject, said wings being fixed to the support on pivot pins making the wings suitable for pivoting between an open position in which the wings clear the access to the support for placing the head of the subject therein and a closed position in which the wings allow fixing the extra-oral means for exerting traction or pushing forces on the transdermal bone anchoring devices (106, 107, 108).

8. The device (1) according to claim 5, characterized in that the connection means for connecting the actuators (104, 105) to the transdermal bone anchoring devices (107, 108) at the rising branches of the mandible comprise rods (114, 115) which extend perpendicularly to the axis of the actuator cylinders and which are provided at their free ends with ball joints (1141, 1151, 1161).

9. The device (1) according to claim 5, characterized in that the connection means between the transdermal bone anchoring device or devices (106) for the anchoring thereof to the mandibular symphysis and the extra-oral means (102, 103) intended for exerting a pushing force on the horizontal branches of the mandible comprise a flexible arcuate strip (113) extending from one of the extra-oral means (102) to the other extra-oral means (103), said strip (113) having an anchoring means (116) for the anchoring thereof to the free end of said transdermal bone anchoring device or devices.

10. The device (1) according to claim 1, characterized in that the device comprises guides (119, 120, 121) for positioning and orienting the head of the subject (BB) against the support (100), such that each transdermal bone anchoring device (106, 107, 108) can be connected with the means (102, 103, 104, 105) for exerting a pushing or traction force for which it is intended.

11. The device (1) according to claim 7, characterized in that it comprises guides (119, 120, 121) for positioning and orienting the head of the subject (BB) against the support (100) and between said wings (109, 110), such that each transdermal bone anchoring device (106, 107, 108) can be connected with the connection means (113, 114, 115, 116) for which it is intended, the guides consisting of two rods (119, 120), each one assembled on a wing (109, 110) and made suitable for sliding with respect to that wing when in the closed position until the end of the rod slightly enters the auditory canal of the subject (BB), and of a third rod (121) suitable for sliding with respect to the support (100) from the top end of that support until the end of the rod (121) abuts against the apex of the head of the subject (BB).

12. The device (1) according to claim 1, characterized in that said support (100) is in the shape of a bassinet, a cradle, a bed or a baby rocker, consisting of a surface for resting the whole rear surface of the subject (BB).

13. The device (1) according to claim 2, characterized in that the device comprises two extra-oral means (104, 105), one (105) for exerting a pushing force on the right rising branch of the mandible by means of at least one transdermal bone anchoring device (107) in said right branch, the other (104) for exerting a pushing force on the left rising branch of the mandible by means of at least one transdermal bone anchoring device (108) in said left branch and two extra-oral means (102, 103) for exerting a pushing force on the two horizontal branches of the mandible by means of one transdermal bone anchoring device (106) in the joining zone of said horizontal branches at the level of the mandibular symphysis.

14. The device (1) according to claim 4, characterized in that each of the extra-oral means (102, 103, 104, 105) for exerting pushing forces comprise a spring actuator.

15. The device (1) according to claim 4, characterized in that each of the extra-oral means (102, 103, 104, 105) for exerting pushing forces comprise-a gas actuator.

16. The device (1) according to claim 2, characterized in that each extra-oral means (102, 103, 104, 105) for exerting a pushing force consists of a spring actuator consisting of a cylinder (1021) fixed on the support (100) by said linking means (109, 110) and housing a rod (1026) and its piston, a compression spring being housed in the cylinder (1021) so as to apply a force suitable for causing an end (1024) of said rod (1026) provided to be connected to at least one transdermal bone anchoring device (106) to come out, a blocking means, the blocking means being a screw (1029) passing through the wall of the cylinder (1021), being provided for blocking the rod (1026) in a specific position.
